(19) United States
(12) Patent Application Publication
(10) Pub. No.: US 2026/0345139 A1
(43) Pub. Date: Nov. 5, 2026

(54) Forward-View Interventional Guidance System

(71) Applicant: The Board of Regents of the University of Oklahoma, Norman, OK (US)

(72) Inventors: Qinggong Tang, Norman, OK (US); Chongle Pan, Norman, OK (US); Chen Wang, Norman, OK (US)

(21) Appl. No.: 19/676,553

(22) Filed: May 13, 2026

Related U.S. Application Data
(63) Continuation-in-part of application No. 17/530,131, filed on Nov. 18, 2021, now abandoned.
(60) Provisional application No. 63/115,452, filed on Nov. 18, 2020.

(51) Int. Cl. A61B 10/04; A61B 5/00; A61B 5/026; A61B 5/20; A61B 34/20

(52) U.S. Cl. CPC A61B 10/04; A61B 5/0066; A61B 5/0084; A61B 5/0261; A61B 5/201; A61B 5/7267; A61B 5/7275; A61B 34/20; A61B 2010/045; A61B 2034/2065; A61B 2562/0242; A61B 2576/00

(57) ABSTRACT

A system comprises a forward-view OCT imaging probe configured to simultaneously acquire structural OCT imaging data and flow-sensitive Doppler OCT imaging data from tissue regions located ahead of and along an insertion axis of the distal tip of the probe. Deep-leaning models process the imaging data to perform tissue recognition, pathological tissue recognition, vascular detection, vascular segmentation, spatial relationship determination, risk assessment, or guidance generation. Structural OCT imaging data identifies different normal tissues, tumor tissues, or other pathological tissues, while Doppler OCT imaging data may be utilized to identify vascular structures and determine distances and sizes of vascular structures relative to the distal tip of the probe based on optical path length information. Guidance information may be generated prior to physical contact between the distal tip and tissue to assist tissue localization, insertion trajectory evaluation, vascular injury avoidance, biopsy guidance, tumor localization, or surgical guidance during interventional procedures.

### CROSS-REFERENCE TO RELATED APPLICATIONS

This is a continuation-in-part of U.S. patent application Ser. No. 17/530,131 filed on Nov. 18, 2021, which claims priority to U.S. Prov. Patent App. No. 63/115,452 filed on Nov. 18, 2020, both of which are incorporated by reference.

### BACKGROUND

Percutaneous nephrostomy (PCN) was first described in 1955 as a minimally invasive, x-ray guided procedure for accessing the renal collecting system in patients with hydronephrosis. PCN needle placement has since become an important clinical technique for establishing access to the renal collecting system for surgical procedures including urinary drainage, urine diversion, and as an initial step for percutaneous nephrolithotomy (PCNL) and other therapeutic interventions, particularly when transurethral access is not feasible. Despite its widespread use, PCN remains technically challenging, as accurate placement of the PCN needle at a target location within the kidney is difficult. During ideal PCN procedure, the needle is advanced through renal tissue layers, including the cortex and medulla, and subsequently passes through a renal calyx before entering the renal pelvis of the collecting system. Precise selection of the PCN needle insertion path is critical to ensure both procedural success and patient safety. Conventional imaging modalities, including ultrasound, fluoroscopy, and computed tomography (CT), are commonly used for PCN needle guidance. However, these modalities generally provide limited spatial resolution and lack sufficient capability to accurately localize the needle tip within tissue. As a result, accurate identification of tissue type at the needle tip and detection of critical structures ahead of the needle remain difficult. It has been reported that single-puncture success rates show substantial variability, and failure rates can reach up to approximately 18%, particularly in complex clinical scenarios. In addition, conventional imaging modalities provide limited capability for differentiating soft tissue structures at the needle tip. For example, fluoroscopy lacks soft tissue contrast and cannot effectively distinguish vascular structures from surrounding tissues. Consequently, inadvertent puncture of renal blood vessels may occur during needle advancement, leading to complications such as hemorrhage. Temporary bleeding has been reported in approximately 95% of PCN cases, with retroperitoneal hematomas occurring in about 13% of cases, and transfusion-requiring hemorrhage in 12-14% of patients treated with PCNL. Furthermore, inaccurate needle placement may lead to other complications, including infection (e.g., fever or sepsis), thoracic complications (e.g., pneumothorax or hydrothorax), and structural injury such as urine leakage or rupture of the pelvicalyceal system. These limitations demonstrate the need for improved guidance techniques capable of providing high-resolution, depth-resolved information at or ahead of the needle tip to enhance needle placement accuracy and reduce complication risks.

Therefore, accurate selection of the insertion route is critical in PCN needle placement. It is generally recommended that the needle be advanced toward the renal calyx area through the calyx papilla, because this pathway typically contains fewer blood vessels and it leads to lower vascular injury risks. However, precise identification of the optimized insertion route remains challenging under clinical situations, even for experienced urologists, due to limited visualization of tissue structures and vascular distribution information along the needle path. In a typical PCN procedure, the needle passes multiple renal tissue layers before reaching the target location. Deviations from the optimal trajectory may lead to unsuccessful needle placement and post-surgical procedures, or tissue damage of vascularized tissue or anatomically complex areas. As a result, multiple needle insertion attempts are often required, further increasing the likelihood of kidney injury, prolonging procedure time, and elevating the risk of complications. Accordingly, there is a critical need for a guidance approach that provides real-time information on tissue type and vascular structures to enable safer trajectory selection and reduce vascular injury risk.

To improve guidance for PCN needle placement, different imaging guidance techniques have been developed. Ultrasound-based methods have been widely utilized due to their safety, accessibility, and real-time imaging capability. For example, contrast-enhanced ultrasound and tracked ultrasonography have been explored to enhance visualization and improve needle guidance. In addition, combined B-mode and Doppler ultrasound techniques have been applied to reduce the risk of hemorrhage by identifying larger blood vessels during needle insertion. Other approaches, including cone-beam computed tomography (CT), retrograde ureteroscopy, and electromagnetic or magnetic field-based navigation systems, have also been employed to improve needle placement accuracy. However, there are still limitations of these techniques for previse needle guidance. These conventional imaging modalities provide limited spatial resolution for accurate tissue recognition or detect small vascular structures at the needle tip. Therefore, reliable assessment of tissue composition and vascular risk during needle advancement remains challenging in clinical practice. Endoscopic approaches have been proposed for PCN needle guidance, wherein an endoscope is integrated within a PCN needle to assist visualization. Although such methods enhance imaging guidance, conventional endoscopic systems based on camera imaging typically provide only surface-level imaging and do not offer depth-resolved information. Consequently, subsurface tissue structures and vascular features located in front of the needle tip cannot be detected prior to physical contact or penetration. In view of the foregoing, a guidance technique capable of providing high-resolution, depth-resolved imaging at and the needle tip is needed to improve trajectory selection and reduce complication risk.

Optical coherence tomography (OCT) is a non-invasive medical imaging modality capable of providing depth-resolved visualization of subsurface tissue structures with penetration depths of several millimeters. By detecting and processing coherent backscattered light from a sample and a reference arm, OCT generates cross-sectional images with micrometer-scale axial resolution, which is significantly higher than that of conventional imaging modalities such as ultrasound, computed tomography (CT), or magnetic resonance imaging (MRI). Due to its high scanning and data acquisition speed, OCT is capable of real-time imaging and volumetric reconstruction through sequential cross-sectional scans. The depth-resolved imaging function of OCT enables recognitions of tissue microstructure based on its optical scattering properties. Structural OCT imaging enables the recognition of different renal tissue layers including cortex, medulla, calyx, sinus fat, and pelvis which demonstrate different tissue microstructures. Furthermore, Doppler OCT enables detection of blood vessel-related signal corresponding to blood flow, thereby allowing identification of vascular structures within kidney tissue. OCT can be implemented in a compact configuration and integrated with fiber-optic probes and endoscopic devices for internal tissue imaging applications. Forward-view endoscopic OCT probes can be configured to acquire imaging data along an insertion axis of a PCN needle, thereby enabling visualization of tissue located ahead of the needle. Such configurations are suitable for interventional procedures in which imaging at or near the instrument tip is required. Despite these advantages, current implementations of OCT in renal applications have been limited. Consequently, there remains a need for an endoscopic OCT system configured to provide structural OCT and Doppler OCT information to enable real-time guidance based on tissue recognition and blood vessel detection functions.

Due to the large volume of imaging data generated during OCT acquisition and variability in subjective operator interpretation, there is a need for automated prediction techniques to enable consistent and efficient analysis to help doctors in surgical guidance. Real-time interpretation of depth-resolved imaging data at PCN needle tip presents practical challenges in clinical settings. Computer-aided methods, including machine learning models such as convolutional neural networks (CNNs), may be utilized to process OCT imaging data to extract clinically relevant information. For example, CNN-based approaches can be configured to analyze structural OCT data to determine tissue classification based on imaging features, and to analyze Doppler OCT data to identify vascular structures based on flow-related signal characteristics. In certain embodiments, such automated processing enables rapid determination of tissue type and spatial relationships between the distal tip of the probe and nearby vascular structures, thereby facilitating generation of guidance information to assist instrument guidance. By reducing reliance on manual interpretation, such approaches can improve consistency, enable real-time decision support, and enhance the safety and efficiency of interventional procedures.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1A is a schematic diagram of a forward-view interventional guidance system.

FIG. 1B is a detailed description of the designed probe head of the forward-view interventional guidance system.

FIG. 2 shows an example workflow for acquiring, processing, labeling, training, validating, and evaluating structural OCT images of different human kidney tissues, and Doppler OCT images of kidney blood vessels from multiple human kidney samples for kidney tissue recognition and kidney blood vessel detection applications, separately.

FIG. 3 shows example structural imaging data and corresponding histological results acquired from different human kidney tissue regions, including cortex, medulla, calyx, sinus fat, and pelvis, for tissue recognition.

FIG. 4 is a table showing tissue recognition performance results by using different deep-learning models for automatic classification and recognition of human kidney tissue types based on structural imaging data.

FIG. 5 shows example Doppler OCT imaging data acquired during insertion of a forward-view interventional guidance system toward human kidney blood vessels of different sizes, together with corresponding histological results.

FIG. 6 shows example automatic segmentation of human kidney blood vessel area and estimations of distance between a forward-view guidance probe needle tip and blood vessel and size of blood vessel on Doppler OCT imaging data acquired during probe insertion, including ground truth labeling, predicted blood vessel information, and intersection-over-union (IoU) evaluation results.

FIG. 7 shows needle placement guidance software based on structural OCT imaging and Doppler OCT imaging results for human kidney tissue recognition and blood vessel detection during PCN needle insertion guidance.

FIG. 8 shows example structural OCT data demonstrating different imaging features between kidney tumors, including malignant carcinoma and benign oncocytoma, and normal kidney tissues.

FIG. 9 is a flowchart illustrating a method showing a process for forward-view interventional guidance during PCN procedure according to one non-limiting embodiment of the present disclosure.

FIG. 10 is a schematic diagram of an apparatus according to one non-limiting embodiment of the present disclosure.

### DETAILED DESCRIPTION

Disclosed herein are embodiments of a forward-view interventional guidance system configured to acquire depth-resolved imaging data from tissue located ahead of a distal tip of an interventional instrument during insertion within a human body. The system comprises a forward-view imaging probe configured to acquire structural OCT imaging data and flow-sensitive Doppler OCT imaging data from tissue regions positioned along an insertion axis extending from the distal tip of the probe. The acquired imaging data may be processed to identify different tissue types, identify tumor or other pathological tissue regions, detect vascular structures, determine spatial relationships between the distal tip of the probe and surrounding anatomical structures, and generate guidance information for assisting needle insertion trajectory selection during an interventional surgical procedure. The guidance information is generated prior to physical contact between the distal tip of the probe and an identified anatomical structure to reduce the risk of tissue injury or vascular rupture during instrument advancement.

In certain embodiments, the disclosed system is configured for use in renal access procedures including PCN, PCNL, renal biopsy procedures, urinary drainage procedures, urine diversion procedures, tumor biopsy procedures, and other interventional procedures involving insertion of an instrument into internal tissue. Structural OCT imaging data acquired from different human kidney tissue and tumor regions, including cortex, medulla, calyx, sinus fat, pelvis, carcinoma tissue, oncocytoma tissue, or combinations thereof, may be processed using one or more trained deep-learning models configured to classify tissue or tumor type based on imaging features including signal intensity distribution, attenuation characteristics, texture distribution, spatial distribution, or morphological patterns. In certain embodiments, the tissue classification information may be utilized to determine whether a current insertion trajectory is approaching a target tissue region, a non-target tissue region, a pathological tissue region, or a non-pathological tissue region during advancement of the interventional instrument.

Flow-sensitive Doppler OCT imaging data may be utilized to identify vascular structures located ahead of the distal tip of the probe during insertion of the interventional instrument. One or more automated segmentation deep learning models may process the acquired imaging data to determine vessel boundaries, vessel size, vessel location, blood-flow-related signal characteristics, or distances between the distal tip of the probe and vascular walls. The determined spatial relationship between the distal tip of the probe and the vascular structures may be continuously updated during insertion to generate real-time guidance information and risk assessment information corresponding to further advancement of the interventional instrument. The generated guidance information may indicate whether to continue insertion along a current trajectory, adjust the insertion trajectory, discontinue advancement, or reposition the interventional instrument toward a target tissue region to reduce a likelihood of vascular injury, sampling error, or incorrect instrument positioning.

The distal tip of the forward-view probe defines a tip-referenced spatial coordinate origin for evaluating tissue structures located ahead of the distal tip during insertion of an interventional instrument. Depth-resolved imaging data acquired from tissue positioned along the insertion axis may be converted into physical distance information relative to the distal tip of the probe based on optical path length, imaging depth calibration, refractive index compensation, or combinations thereof. Accordingly, tissue structures and vascular structures identified within the imaging field may be spatially localized relative to the distal tip of the probe during advancement of the interventional instrument. In certain embodiments, the spatial relationship between the distal tip of the probe and surrounding tissue structures may be continuously updated in real time to generate guidance information corresponding to insertion trajectory selection, target tissue localization, vascular injury avoidance, or combinations thereof.

In certain embodiments, the disclosed system may generate guidance information prior to physical contact between the distal tip of the probe and tissue to assist tissue localization, insertion trajectory evaluation, vascular injury avoidance, target tissue identification, biopsy guidance, or combinations thereof during advancement of the interventional instrument.

Structural OCT imaging data and flow-sensitive Doppler OCT imaging data may be processed together to evaluate both tissue composition and vascular risk during the interventional instrument advancement. Structural OCT imaging data may be utilized to identify normal tissue regions, tumor and other pathological tissue regions, target tissue regions, non-target tissue regions, or combinations thereof, while flow-sensitive Doppler OCT imaging data may be utilized to identify blood-flow-related signal characteristics corresponding to vascular structures.

In certain embodiments, one or more image classification deep-learning models, image segmentation models, image-processing algorithms, or combinations thereof may be utilized to process the acquired imaging data. The one or more deep-learning models may comprise one or more CNNs configured to classify tissue type, identify pathological tissue, detect vascular structures, segment blood vessel regions, estimate spatial relationships, generate risk assessment information, or combinations thereof.

In certain embodiments, the disclosed system may be utilized during renal access procedures including PCN, PCNL, renal biopsy procedures, tumor biopsy procedures, urinary drainage procedures, urine diversion procedures, or combinations thereof. The disclosed system may further be utilized for guidance of other interventional procedures involving insertion of an instrument into internal tissue, including localized surgery guidance, tumor localization procedures, vascular avoidance procedures, drug delivery procedures, anesthesia needle guidance procedures, or combinations thereof.

The disclosed systems and methods may be implemented using hardware, software, firmware, deep-learning architectures, image-processing algorithms, or combinations thereof. The disclosed embodiments are not limited to a particular imaging architecture, neural-network architecture, processing workflow, imaging parameter, insertion geometry, clinical implementation, or interventional procedure unless expressly stated otherwise.

The following abbreviations apply:
BD: balanced detector
CNN: convolutional neural network
CPU: central processing unit
CT: computed tomography
DAQ: data acquisition
FC: fiber coupler
FOV: field of view
GRIN: gradient-index
GSM: galvanometer scanning mirror
IoU: intersection-over-union
mm: millimeter(s)
MRI: magnetic resonance imaging
MZI: Mach-Zehnder interferometer
OCT: optical coherence tomography
PC: polarization controller
PCN: percutaneous nephrostomy
PCNL: percutaneous nephrolithotomy
2D: two-dimensional
3D: three-dimensional

Before describing various embodiments of the present disclosure in more detail by way of exemplary description, examples, and results, it is to be understood that the present disclosure is not limited in application to the details of methods and compositions as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-70, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. A reference to degrees such as 1 to 90 is intended to explicitly include all degrees in the range.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” are used to indicate that a value includes the inherent variation of error. Further, in this detailed description, each numerical value (e.g., degrees or frequency) should be read once as modified by the term “about” (unless already expressly so modified), and then read again as not so modified unless otherwise indicated in context. As noted, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. The use of the term “about” may mean a range including ±10% of the subsequent number unless otherwise stated.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement (e.g., degrees, frequency, width, length, etc.).

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

### System

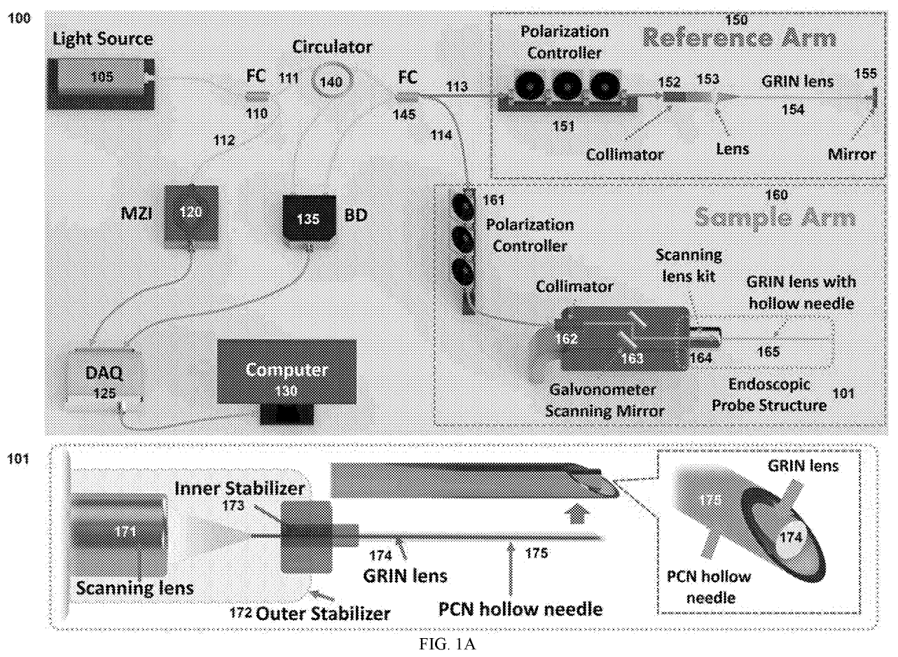

FIG. 1A is a schematic diagram of a forward-view interventional guidance system 100 according to one non-limiting embodiment of the present disclosure, and FIG. 1B illustrates a detailed configuration of a forward-view probe head of the forward-view interventional guidance system 100. The forward-view interventional guidance system 100 is configured to acquire depth-resolved imaging data from tissue located ahead of a distal tip of an interventional instrument during insertion within tissue. In certain embodiments, the disclosed system is configured for use in interventional procedures including PCN, PCNL, renal biopsy procedures, tumor biopsy procedures, urinary drainage procedures, urine diversion procedures, localized surgery procedures, anesthesia needle guidance procedures, drug delivery procedures, or combinations thereof. The disclosed system may be configured to provide real-time tissue recognition, pathological tissue localization, vascular detection, insertion trajectory evaluation, spatial localization, risk assessment, or combinations thereof during interventional instrument advancement within tissue.

The forward-view interventional guidance system 100 comprises an optical coherence tomography (OCT) imaging system configured to acquire structural OCT imaging data and flow-sensitive Doppler OCT imaging data from tissue regions located ahead of the distal tip of the interventional instrument. In certain embodiments, the OCT imaging system comprises a SS-OCT system. The forward-view interventional guidance system 100 may comprise an endoscopic probe structure 101, a light source 105, a first fiber coupler (FC) 110, one or more optical paths 111-114, a Mach-Zehnder interferometer (MZI) 120, a data acquisition (DAQ) board 125, a computer 130, a balanced detector (BD) 135, a circulator 140, a second FC 145, a reference arm 150, a sample arm 160, a reference arm polarization controller (PC) 151, a reference arm collimator 152, a reference arm optical lens 153, a reference arm gradient index (GRIN) rod lens 154, a reference arm mirror 155, a sample arm PC 161, a sample arm collimator 162, a galvanometer scanning mirror set 163, a scanning lens kit 164, and a combination of a sample arm GRIN rod lens with a hollow needle 165. The disclosed system is configured to generate depth-resolved imaging information from tissue positioned ahead of the distal tip of the probe during advancement of the interventional instrument.

The endoscopic probe structure 101 is positioned at a distal portion of the scanner body 102 and is configured to support one or more optical and mechanical components of the forward-view probe head. The scanning lens kit 164 may comprise a scanning lens 171, an outer stabilizer 172, an inner stabilizer 173, and a mounting fixture 176. The outer stabilizer 172 is secured to a distal portion of the scanner body 102, and the inner stabilizer 173 is configured to threadedly engage the outer stabilizer 172. The combination of the sample arm GRIN lens with the hollow needle 165 comprises a sample arm GRIN rod lens 174 and a hollow needle 175 configured to provide forward-view imaging capability during the interventional instrument insertion. The galvanometer scanning mirror set 163 may be electrically controlled through an electric mirror control cable 166.

The disclosed system further comprises a mounting fixture 176 configured to secure one or more optical components of the forward-view probe head. The mounting fixture 176 comprises upper and lower mirror-symmetric clamping structures configured to secure the combination of the sample arm GRIN lens with the hollow needle 165. The mounting fixture 176 is secured using one or more screws 177 and one or more nuts 178. The disclosed mounting configuration assists positioning, stabilization, alignment, fixation, or combinations thereof of the forward-view probe head components during operation.

The disclosed forward-view probe head enables optical scanning performed at a proximal end of the sample arm GRIN rod lens 174 to be optically relayed toward tissue located ahead of a distal end of the sample arm GRIN rod lens 174 while maintaining a miniaturized probe geometry suitable for insertion into tissue through the hollow needle 175. Dimensions of the sample arm GRIN rod lens 174 and corresponding mounting fixtures 176 may be selected according to a target interventional procedure utilizing a corresponding hollow interventional instrument. Different combinations of GRIN rod lenses and mounting fixtures may be utilized for compatibility with different needles, trocars, catheters, biopsy instruments, nephrostomy instruments, epidural cannulas, or combinations thereof utilized in different interventional procedures. The disclosed forward-view probe head enables acquisition of depth-resolved imaging data from tissue positioned ahead of the distal tip of the probe along an insertion axis of the interventional instrument.

The sample arm GRIN lens 154 defines a forward-view optical relay configuration configured to preserve spatial relationships between proximal optical scanning components and tissue structures located ahead of the distal tip of the probe. The proximal surface of the sample arm GRIN lens 154 may be positioned adjacent to a focal plane of an optical scanning component such that optical scanning performed at a proximal end of the system is optically relayed toward tissue positioned ahead of the distal tip of the probe. Positioning of the proximal surface of the sample arm GRIN lens 154 relative to the focal plane may be adjusted through threaded engagement between the outer stabilizer 172 and the inner stabilizer 173, wherein adjustment of a threaded engagement depth enables optical alignment and focal positioning of the sample arm GRIN lens 154. The disclosed forward-view optical relay configuration enables tip-referenced imaging and spatial localization of tissue structures during advancement of the interventional instrument along the insertion axis.

The distal tip of the forward-view probe defines a tip-referenced spatial coordinate origin for evaluating tissue structures located ahead of the distal tip during the interventional instrument insertion. Depth-resolved imaging data acquired from tissue positioned along the insertion axis may be converted into physical distance information relative to the distal tip of the probe based on optical path length, imaging depth calibration, refractive index compensation, or combinations thereof. Accordingly, tissue structures and vascular structures identified within the imaging field may be spatially localized relative to the distal tip of the probe during the interventional instrument advancement.

The light source 105 comprises a broadband laser source configured to generate an optical signal for OCT imaging. The optical signal may be divided by a first FC 110 into a circulator 140 utilized for OCT imaging through a first optical path 111, and an MZI 120 for OCT sampling synchronization through a second optical path 112. In certain embodiments, the circulator 140 is configured to direct the optical signal from the first FC 110 to the second FC 145. The second FC 145 is configured to divide the optical signal into a reference arm 150 through the third optical path 113, and the sample arm 160 through the fourth optical path 114. Backscattered optical signals returning from the reference arm 150 and the sample arm 160 are directed by the circulator 140 and the second FC 145 toward the BD 135 for interferometric detection. The optical signal propagating through the sample arm 160 is directed toward tissue located ahead of the distal tip of the forward-view probe, while the optical signal propagating through the reference arm 150 is utilized for interferometric signal generation. In the sample arm 160, the optical signal propagates through the sample arm PC 161 for polarization adjustment and noise reduction, and subsequently passes through the sample arm collimator 162 toward the scanner body 102. The optical signal is directed to the galvanometer scanning mirror (GSM) set 163 to perform optical scanning of different spatial positions. The scanning lens kit 164 comprises the scanning lens 171 configured to focus and direct the optical signal toward a proximal end of the combination of the sample arm GRIN rod lens with the hollow needle 165. The optical signal is optically relayed through the sample arm GRIN rod lens 174 to acquire depth-resolved imaging information from tissue located ahead of a distal end of the sample arm GRIN rod lens 174 during insertion of the interventional instrument. Backscattered optical signals acquired from tissue located ahead of the distal end propagate back through the sample arm 160 for interferometric detection. In the reference arm 150, the optical signal propagates through the reference arm PC 151 and subsequently through the reference arm collimator 152. The collimated optical signal passes through the reference arm optical lens 153 and is directed into the reference arm GRIN rod lens 154. The reference arm GRIN rod lens 154 is configured to compensate optical dispersion associated with the sample arm 160 and maintain interferometric matching between the reference arm 150 and the sample arm 160. The reference optical signal is reflected by the reference arm mirror 155 and subsequently combined with backscattered optical signals returning from the sample arm 160 to generate interferometric signals for OCT imaging. Backscattered optical signals acquired from tissue located ahead of the distal tip interfere with the reference optical signal and are subsequently detected by the BD 135 and processed by the computer 130 and the DAQ board 125 to generate depth-resolved imaging information.

The generated imaging information comprises structural OCT imaging data and flow-sensitive Doppler OCT imaging data. Structural OCT imaging data may be processed to obtain tissue morphology information, spatial tissue information, tissue classification information, pathological tissue information, or combinations thereof. In certain embodiments, structural OCT imaging data may be utilized to identify different tissue regions including cortex, medulla, calyx, sinus fat, pelvis, malignant and benign tumor tissues, or combinations thereof. Flow-sensitive Doppler OCT imaging data may be processed to obtain blood-flow-related information, vascular structure information, vessel boundary information, vessel size information, vessel location information, distances between the distal tip of the probe and vascular structures, or combinations thereof.

In certain embodiments, one-dimensional scanning or two-dimensional scanning may be performed to generate two-dimensional (2D) imaging data, three-dimensional (3D) imaging data, or combinations thereof. The disclosed system may further comprise miniaturized optical components configured for insertion into a hollow interventional instrument while maintaining forward-view imaging capability. Imaging performance parameters including axial resolution, lateral resolution, imaging sensitivity, imaging speed, imaging depth, field-of-view (FOV), probe diameter, probe length, viewing angle, or combinations thereof may vary according to a target interventional procedure, dimensions of a hollow interventional instrument, imaging configuration, optical design, or combinations thereof. In certain embodiments, dimensions of the sample arm GRIN lens 174 may be selected according to dimensions of a needle, trocar, catheter, biopsy instrument, nephrostomy instrument, epidural cannulas, or combinations thereof utilized in a target procedure.

In certain embodiments, the disclosed system generates guidance information corresponding to advancement of the interventional instrument within tissue. The guidance information may comprise visual guidance information, graphical risk information, target tissue indication information, vascular warning information, trajectory adjustment information, insertion recommendation information, or combinations thereof. The guidance information assists localization of target tissue regions and reduction of vascular injury risk during advancement of the interventional instrument.

Structural OCT imaging data and Doppler OCT imaging data may be processed together to evaluate tissue composition and vascular structures during insertion of the interventional instrument. One or more deep-learning models, image-processing algorithms, segmentation models, or combinations thereof may process the acquired imaging data to classify tissue type, identify pathological tissue regions, detect vascular structures, determine spatial relationships between tissue structures and the distal tip of the probe, generate guidance information, or combinations thereof. In certain embodiments, the one or more deep-learning models comprise one or more convolutional neural networks (CNNs), image segmentation networks, classification networks, or combinations thereof configured to process imaging features including signal intensity distributions, attenuation characteristics, spatial texture distributions, morphological patterns, flow-related signal characteristics, phase-related signal characteristics, frequency-related signal characteristics, or combinations thereof.

### Imaging and Processing Workflow

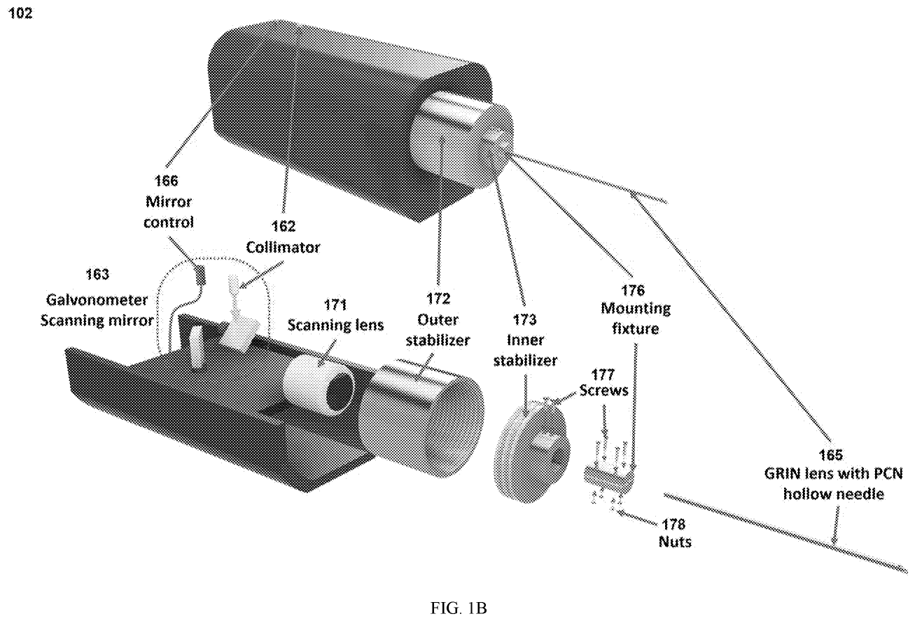

FIG. 2 illustrates a representative imaging and processing workflow 200 for acquisition, processing, labeling, training, validation, evaluation, tissue recognition, vascular detection, or combinations thereof using structural OCT imaging data and Doppler OCT imaging data acquired from human kidney tissue samples. Structural OCT imaging data acquisition 210 may be realized from different tissue regions including cortex, medulla, calyx, sinus fat, pelvis, or combinations thereof. Flow-sensitive Doppler OCT imaging data acquisition 220 may be realized from vascular structures located within tissue.

Automatic tissue recognition and vascular detection may be performed based on the acquired imaging data following data acquisition. The acquired imaging data may be processed using image preprocessing, signal-region selection, image normalization, image cropping, feature extraction, image labeling, segmentation, or combinations thereof. Histological analysis may be utilized as reference information for correlating imaging characteristics with corresponding tissue structures. The processed imaging data may be utilized to train, validate, and evaluate one or more deep-learning models configured for automatic tissue classification and vascular segmentation using cross-validation, cross-testing, and external testing procedures to evaluate deep learning model generalization and prediction performance.

### Structural OCT Imaging of Tissue Regions and Automatic Tissue Recognition

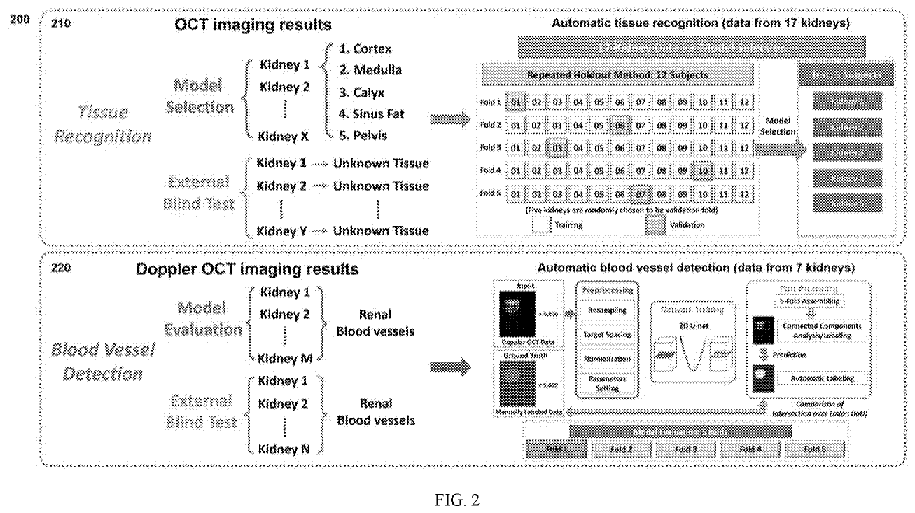

FIG. 3 illustrates representative structural OCT imaging data acquired from different human kidney tissue regions using the disclosed forward-view interventional guidance system 100 under structural OCT scanning mode 305. In certain embodiments, different tissue regions include cortex, medulla, calyx, sinus fat, and pelvis. Representative 3D OCT imaging data 310, 320, 330, 340, and 350, corresponding 2D cross-sectional OCT imaging data 315, 325, 335, 345, and 355, and corresponding histological results 361-364 are shown for comparison of different tissue structures.

In certain embodiments, different tissue regions demonstrate distinguishable structural OCT imaging characteristics beneath the superficial bright reflection on the top corresponding to the GRIN lens interface suitable for automatic tissue recognition. Cortex tissue and medulla tissue may demonstrate relatively homogeneous scattering distributions with different imaging depths and signal attenuation characteristics. Calyx tissue may demonstrate layered structural features and relatively strong superficial signal intensity distributions. Sinus fat tissue may demonstrate heterogeneous punctate scattering patterns corresponding to adipose tissue structures. Pelvis tissue may demonstrate little to no structural OCT signal beneath the GRIN lens due to the cavity-like anatomical structure of the renal pelvis region. These distinguishable structural OCT imaging features may be utilized for automatic tissue classification using one or more deep-learning models.

Structural OCT imaging data acquired from different tissue regions may be processed to generate tissue classification information during advancement of the interventional instrument. The disclosed tissue-recognition workflow may enable automatic differentiation of cortex, medulla, calyx, sinus fat, pelvis, or combinations thereof based on imaging features including signal intensity distributions, attenuation characteristics, spatial texture distributions, morphological patterns, layered structures, scattering characteristics, or combinations thereof. The generated tissue classification information may be utilized for tissue localization, insertion trajectory evaluation, target tissue identification, guidance generation, or combinations thereof during interventional procedures.

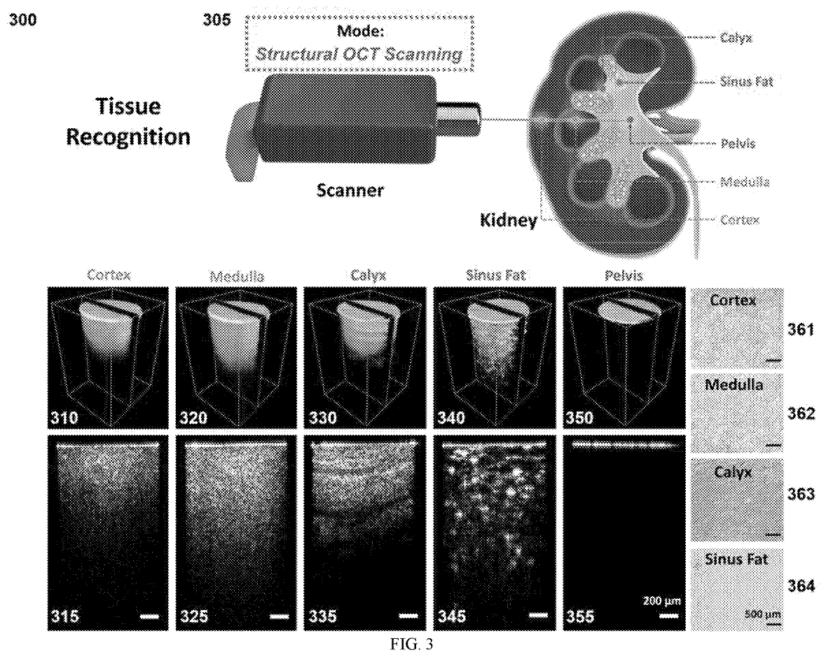

FIG. 4 illustrates representative automatic tissue-recognition performance results 400, including prediction results using three deep-learning models 410, confusion matrix 420, and statistical evaluation results 430 associated with the selected model, configured to process structural OCT imaging data acquired from different human kidney tissue regions. The disclosed deep-learning models may differentiate cortex tissue, medulla tissue, calyx tissue, sinus fat tissue, pelvis, or combinations thereof based on imaging features including signal intensity distributions, attenuation characteristics, spatial texture distributions, layered structures, scattering characteristics, or combinations thereof. The disclosed automatic tissue-recognition framework enables real-time tissue classification and guidance generation during the interventional instrument advancement.

### Flow-Sensitive Doppler OCT Imaging of Blood Vessels and Automatic Vascular Segmentation and Evaluation

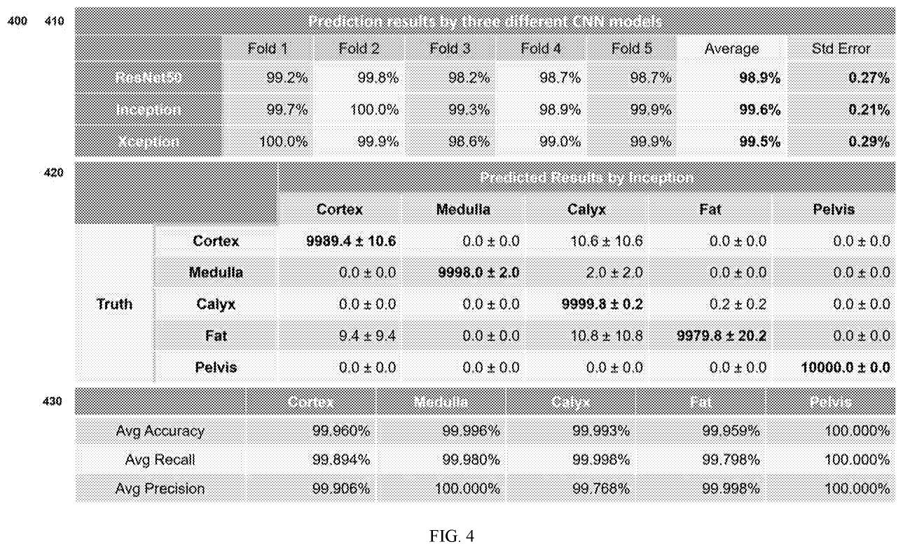

FIG. 5 illustrates representative flow-sensitive Doppler OCT imaging data 500 acquired from renal blood vessels located ahead of the distal tip of the forward-view interventional guidance system 505 under Doppler OCT scanning mode. Representative flow-sensitive Doppler OCT imaging results of blood vessels with different sizes including small 510, medium 520, and large 530 with corresponding histological results 541-543 are shown for comparison of vascular structures. Doppler OCT imaging enables visualization of blood-flow-related signal characteristics corresponding to vascular structures located ahead of the distal tip during advancement of the interventional instrument. As the distal tip of the probe approaches a blood vessel, the distance between the distal tip and the vascular structure decreases and corresponding Doppler OCT imaging features may continuously change during insertion. The disclosed Doppler OCT imaging configuration may enable identification of small blood vessels located ahead of the distal tip during advancement of the interventional instrument.

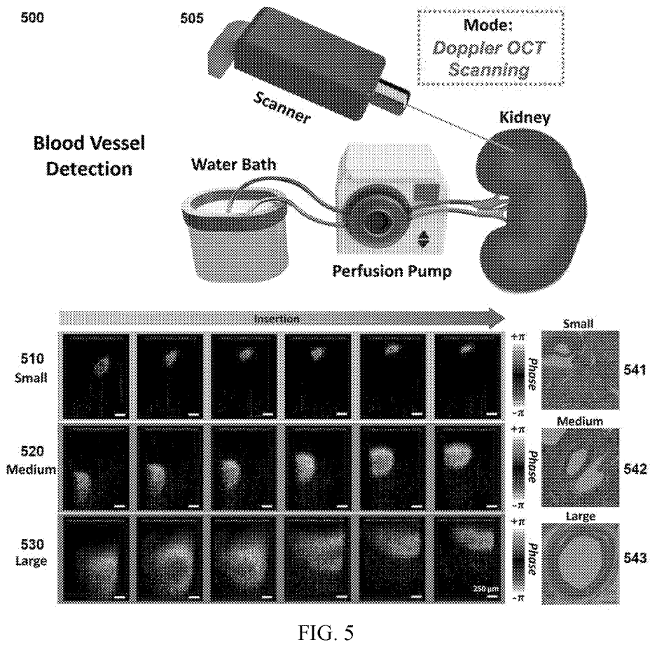

FIG. 6 illustrates representative automatic vascular segmentation results and corresponding evaluation information 600 based on Doppler OCT imaging data. Original Doppler OCT results 610, Ground-truth vascular labeling results 620, and corresponding predicted vascular segmentation and evaluation results 630 are shown for representative blood vessel segmentation embodiments. In certain embodiments, deep-learning models such as nnU-Net architecture may process Doppler OCT imaging data to identify vascular boundaries, vessel regions, vessel size, distances between the distal tip of the probe and vascular structures, or combinations thereof. Overlap evaluations between predicted segmentation regions and corresponding ground-truth vascular regions 640 or background regions 650 may be evaluated using intersection-over-union (IoU) analysis.

The predicted vascular segmentation results and corresponding evaluation information may be utilized to assist the interventional instrument advancement. The disclosed vascular segmentation and evaluation framework may enable automatic identification of blood vessels located ahead of the distal tip of the probe and generation of guidance information corresponding to vascular injury avoidance, insertion trajectory evaluation, risk assessment, advancement recommendation, or combinations thereof during interventional procedures.

### Guidance Platform Interface

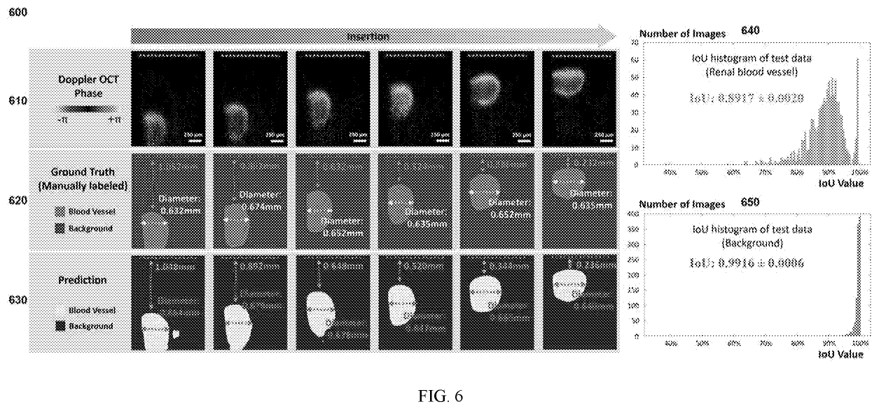

FIG. 7 illustrates a representative guidance platform interface 700 configured to display structural OCT imaging data with tissue recognition results, and flow-sensitive Doppler OCT imaging data with vascular segmentation information during the interventional instrument advancement. The disclosed guidance platform interface may provide real-time visualization of tissue regions and vascular structures located ahead of the distal tip of the forward-view probe to assist tissue localization, insertion trajectory evaluation, vascular injury avoidance, target tissue identification, advancement recommendation, or combinations thereof.

### Tumor Detection Embodiments

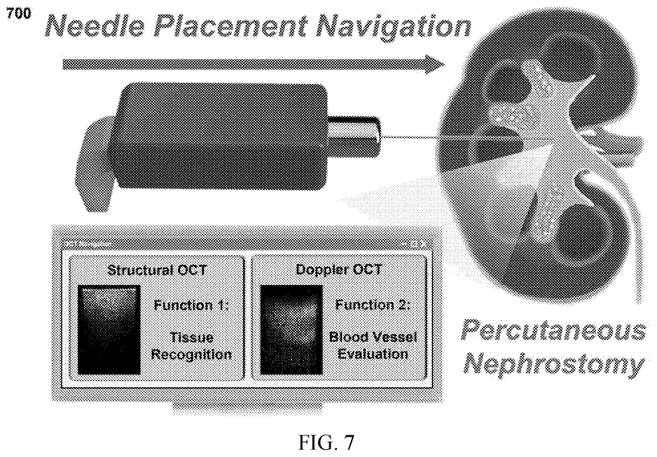

FIG. 8 illustrates representative structural OCT imaging data acquired from kidney tumors and normal kidney tissue 800 during tissue biopsy 810 using the disclosed forward-view interventional guidance system. Malignant tumor tissue, benign tumor tissue, and normal tissue may demonstrate different structural OCT imaging characteristics suitable for tissue differentiation and tumor or other pathological tissue recognition. In certain embodiments, malignant carcinoma tumor tissue and benign oncocytoma tumor tissue may demonstrate more heterogeneous and irregular scattering distributions compared to normal kidney tissue. Compared to benign oncocytoma, malignant carcinoma may demonstrate stronger signal intensity distributions and reduced imaging depth. The disclosed OCT imaging framework may enable identification of tumor or other pathological tissue and normal tissue, differentiation of benign tumor tissue and malignant tumor tissue, or combinations thereof to assist tumor localization, biopsy guidance, tissue targeting, surgical guidance, interventional decision-making, or combinations thereof during interventional procedures.

### Exemplary Method

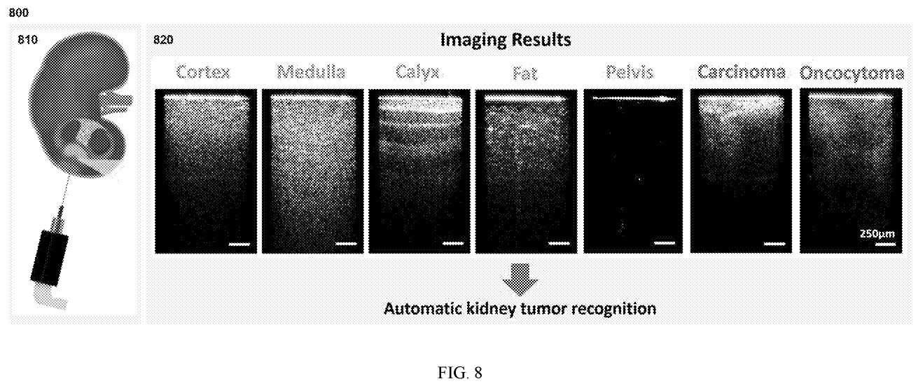

FIG. 9 illustrates a representative forward-view interventional guidance method 900 using the disclosed forward-view interventional guidance system during an interventional procedure. At step 910, a GRIN lens endoscope configured for compatibility with a hollow interventional needle utilized in a target surgical procedure may be obtained. At step 920, a hollow interventional needle utilized in the interventional procedure may be obtained. At step 930, the GRIN lens endoscope may be integrated into the hollow interventional needle to form a forward-view interventional guidance system. At step 940, the forward-view interventional guidance system may be inserted into a human body toward a target tissue region. At step 950, structural OCT imaging data and flow-sensitive Doppler OCT imaging data may be simultaneously acquired from tissue regions located ahead of and along an insertion axis of the distal tip of the probe during advancement of the forward-view interventional guidance system within tissue. Deep-learning models may process the acquired imaging data to recognize tissue regions, tumor or other pathological tissue regions, vascular structures, spatial relationships between the distal tip and the vascular structures, or combinations thereof. The spatial relationships may comprise distances and sizes of vascular structures relative to the distal tip based on optical path length information derived from the acquired imaging data. The acquired imaging data may be evaluated under one or more predefined conditions to generate guidance information, risk assessment information, insertion trajectory evaluation information, advancement recommendations, or combinations thereof prior to physical contact between the distal tip of the probe and the tissue regions. At step 960, the GRIN lens endoscope may be withdrawn from the human body while leaving the hollow interventional needle positioned within the human body for a subsequent interventional or surgical procedure.

### Exemplary Computing Apparatus

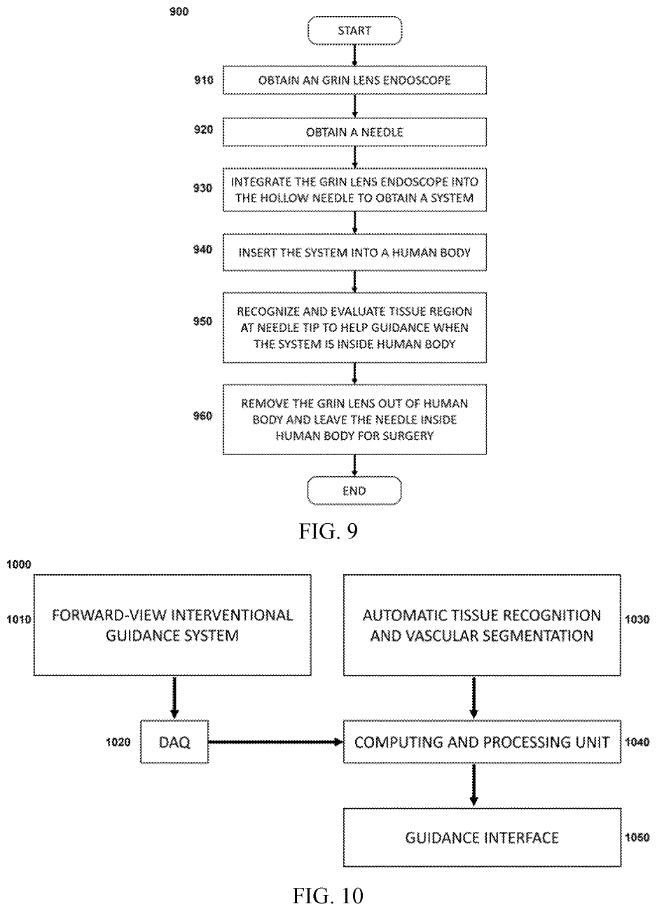

FIG. 10 is a schematic diagram of an apparatus 1000 according to one non-limiting embodiment of the present disclosure. The apparatus 1000 may implement one or more disclosed embodiments associated with the forward-view interventional guidance system. The apparatus 1000 may comprise a forward-view interventional guidance system 1010, a DAQ 1020, an automatic tissue recognition and vascular segmentation module 1030, a computing and processing unit 1040, and a guidance interface 1050.

The DAQ interface 1020 may acquire structural OCT imaging data and flow-sensitive Doppler OCT imaging data from the forward-view interventional guidance system 1010 and transmit the acquired imaging data to the computing and processing unit 1040. The automatic tissue recognition and vascular segmentation module 1030 may comprise deep-learning models configured to perform automatic tissue recognition and vascular segmentation based on the acquired imaging data.

The computing and processing unit 1040 may comprise one or more central processing units (CPUs), graphical processing units (GPUs), memory devices, storage devices, and other electronic devices configured to process, store, analyze, display, or evaluate structural OCT imaging data, flow-sensitive Doppler OCT imaging data to assist automatic tissue recognition and vascular segmentation during advancement of the interventional instrument.

The guidance interface 1050 may provide real-time visualization of tissue regions, tumor or other pathological tissue regions, vascular structures, spatial relationships, insertion trajectories, risk assessment information, guidance information, or combinations thereof to assist tissue localization, target tissue identification, vascular injury avoidance, biopsy guidance, surgical guidance, or combinations thereof during interventional procedures.

Non-limiting embodiments of the present disclosure include but are not limited to those shown below.
Embodiment 1. A method comprising: a tip-referenced forward-view interventional guidance method for navigating an instrument within tissue, comprising: inserting a probe into biological tissue of an internal organ of a subject, the probe having a distal tip; acquiring depth-resolved structural optical coherence tomography (OCT) and flow-sensitive Doppler OCT data simultaneously from a region located ahead of and along an insertion axis of the distal tip while the probe is positioned within the biological tissue of an internal organ of a subject; determining, using the depth-resolved structural OCT data, a tissue classification of a region ahead of the distal tip relative to surrounding tissues based on imaging features; identifying a specific tissue type, tumor or other pathological tissue types, or combinations thereof based on the tissue classification; determining, using the depth-resolved Doppler OCT data, a spatial relationship between the one or more vascular structures and the distal tip, the spatial relationship including at least (i) a distance from the distal tip to the one or more vascular structures, and (ii) a size of the one or more vascular structures; evaluating the spatial relationship in view of the identified specific tissue type and a predefined condition; generating guidance information based on the evaluation, wherein the guidance information is configured to adjust a trajectory or position of the probe relative to both of the tissue type and the one or more vascular structures at the probe distal tip to assist tissue targeting, biopsy guidance, or combinations thereof, wherein the tissue classification is performed exclusively based on the structural OCT data, and the one or more vascular structures are identified exclusively based on the Doppler OCT data; wherein the distance from the distal tip is determined based on an optical path length corresponding to a depth position along the insertion axis originating from the distal tip of the probe; wherein the region ahead of the distal tip is imaged prior to physical contact such that the guidance information is generated before the distal tip reaches the region; and wherein evaluating the spatial relationship comprises comparing the distance and size of the one or more vascular structures to a threshold that varies according to the identified specific tissue type to generate a risk metric, and the guidance information is generated based on the risk metric.
Embodiment 2. The method of embodiment 1, wherein determining the tissue classification comprises: processing the depth-resolved structural OCT data using a trained deep learning model trained on labeled OCT data corresponding to known tissue types of the internal organ, the model being configured to classify tissue types based on imaging features including at least one of signal intensity distribution, attenuation characteristics, or morphological features derived from the depth-resolved structural OCT data.
Embodiment 3. The method of embodiment 1, wherein identifying the one or more vascular structures comprises: analyzing Doppler OCT signal variations including phase shift or frequency shift features indicative of moving scatterers corresponding to blood flow.
Embodiment 4. The method of embodiment 1, wherein the structural OCT data and Doppler OCT data are acquired simultaneously and correspond to a same spatially co-registered forward-view imaging region ahead of the probe distal tip.
Embodiment 5. The method of embodiment 1, wherein the distance from the distal tip is determined relative to a reference position defined at the distal tip, by converting optical path length information derived from the depth-resolved imaging data into a physical distance along the insertion axis, such that the distance corresponds to a spatial location ahead of the distal tip of the probe.
Embodiment 6. The method of embodiment 1, wherein the predefined condition is dependent on the identified specific tissue type.
Embodiment 7. The method of embodiment 6, wherein the predefined condition includes a risk threshold that varies according to the identified specific tissue type.
Embodiment 8. The method of embodiment 1, further comprising: generating a risk evaluation result representing a probability or likelihood of encountering a vascular structure within a predefined distance ahead of the distal tip based on the structural OCT data and the Doppler OCT data.
Embodiment 9. The method of embodiment 1, wherein generating the guidance information comprises determining an adjusted insertion trajectory that reduces a likelihood of intersecting the one or more vascular structures based on the evaluated risk metric.
Embodiment 10. The method of embodiment 1, wherein the acquiring, identifying, determining, and generating steps are performed continuously in real time during insertion of the probe.
Embodiment 11. The method of embodiment 1, wherein the identified tissue type comprises a pathological tissue region including benign tumor tissue or malignant tumor tissue.
Embodiment 12. The method of embodiment 11, wherein generating the guidance information comprises generating biopsy guidance information based on differentiation between tumor or other pathological tissues and surrounding normal tissues.
Embodiment 13. The method of embodiment 1, wherein the gradient index (GRIN) lens is disposed at and defines the distal tip to provide forward-view imaging at the distal tip, the GRIN lens being configured to directly interface with tissue, and wherein the probe is free of any optical scanning component at the distal tip such that all optical scanning is performed at a proximal location.
Embodiment 14. The method of embodiment 13, wherein optical scanning is performed by a scanning mechanism located proximally to the probe, and wherein the GRIN lens relays scanned optical beams from the proximal scanning mechanism to the distal tip.
Embodiment 15. The method of embodiment 14, wherein the GRIN lens is optically coupled to the proximal scanning mechanism via at least one of an optical fiber, a fiber-optic collimator, or a coupling adapter.
Embodiment 16. The method of embodiment 1, wherein the probe is configured to be removably inserted within a lumen of a needle, trocar, catheter, cannula, biopsy instrument, or combinations thereof.
Embodiment 17. The method of embodiment 13, wherein the GRIN lens is configured to provide a forward-view imaging field aligned with an insertion axis of the probe.
Embodiment 18. The method of embodiment 13, wherein the probe is free of any motorized or electromechanical component at the distal tip.
Embodiment 19. The method of embodiment 13, wherein the GRIN lens has a pitch and length selected to position an imaging plane at or near the distal tip of the probe.
Embodiment 20. A tip-referenced forward-view interventional guidance system for navigating an instrument within tissue, comprising: a probe configured to be inserted into tissue and having a distal tip, the probe being configured to acquire depth-resolved structural OCT data and Doppler OCT data simultaneously in real time from a region located ahead of and along an insertion axis of the distal tip positioned within the biological tissue of an internal organ of a subject; a computing and processing unit configured to: (i) determine, using the structural OCT data, a tissue classification including normal tissues and tumor or other pathological tissues of a region ahead of the distal tip of the probe based on depth-resolved optical signal characteristics; (ii) identify one or more vascular structures based on flow-related information derived from the Doppler OCT data; (iii) determine a spatial relationship between the one or more vascular structures and the distal tip, including at least a distance and a size relative to the distal tip, wherein the distance is determined by converting optical path length information derived from the depth-resolved OCT data into a physical distance referenced to the distal tip of the probe along the insertion axis; (iv) evaluate the spatial relationship in view of the tissue classification under a predefined condition to determine a risk metric associated with advancing the distal tip toward the one or more vascular structures prior to physical contact, and generate guidance information based on the risk metric; wherein the guidance information is configured to adjust a trajectory or position of the probe relative to the one or more vascular structures.
Embodiment 21. The system of embodiment 20, wherein the probe comprises a GRIN lens disposed at a forward portion of the probe and extending to the distal tip, the distal end of the GRIN lens defining an imaging interface at the distal tip at which forward-view imaging is performed directly into tissue ahead of the distal tip, and wherein optical scanning is performed by a scanning mechanism located proximal to the probe such that the probe is free of any optical or electromechanical scanning component at the distal tip and is configured to transmit scanned light from the proximal scanning mechanism to the distal tip through the GRIN lens.
Embodiment 22. The system of embodiment 20, wherein the probe is configured to be removably inserted within a lumen of a needle or trocar such that the probe provides real-time guidance during advancement of the needle or trocar within tissue prior to tissue sampling, and is withdrawable after positioning while the needle or trocar remains in place to perform a subsequent tissue sampling procedure without additional insertion.
Embodiment 23. The system of embodiment 20, wherein the probe is configured to acquire the depth-resolved imaging data from tissue located ahead of the distal tip prior to physical contact between the distal tip and the tissue, such that the imaging data represents subsurface tissue structure at a distance from the distal tip based on optical path length information.
Embodiment 24. The system of embodiment 20, wherein the spatial relationship is determined in a coordinate system referenced to the distal tip of the probe and aligned with the insertion axis such that distances are measured relative to the distal tip along the insertion direction.
Embodiment 25. The system of embodiment 20, wherein identifying one or more vascular structures comprises detecting flow-related frequency features in Doppler OCT signals, the flow-related frequency features corresponding to motion-induced phase shifts indicative of blood flow.
Embodiment 26. The system of embodiment 20, wherein the predefined condition is determined based on the tissue classification and defines a risk metric associated with vascular injury as a function of a distance to and a size of the one or more vascular structures.
Embodiment 27. The system of embodiment 20, wherein the spatial relationship is dynamically and continuously updated based on successive acquisitions of the depth-resolved imaging data as the probe advances within the tissue.
Embodiment 28. The system of embodiment 20, wherein the guidance information is generated in real time during insertion of the probe with a latency sufficiently low to enable continuous adjustment of the probe trajectory.
Embodiment 29. The system of embodiment 20, wherein the guidance information is configured to assist an operator in adjusting a trajectory of a needle during insertion by providing visual, auditory, or haptic feedback indicative of the spatial relationship and the associated risk metric.
Embodiment 30. The system of embodiment 20, wherein the guidance information is configured to automatically control movement of the probe to adjust the trajectory relative to the one or more vascular structures based on the determined spatial relationship and the risk metric.
Embodiment 31. The system of embodiment 20, wherein the computing and processing unit is configured to differentiate benign tumor tissue from malignant tumor tissue based on structural OCT imaging characteristics.
Embodiment 32. The system of embodiment 20, wherein the guidance information comprises biopsy guidance information associated with a pathological tissue region.
Embodiment 33. A tip-referenced forward-view imaging guidance assembly configured to be inserted into a lumen of an instrument, comprising: an optical probe having a distal end that defines a distal tip and configured to acquire depth-resolved structural OCT data and Doppler OCT data simultaneously in real time from a region located ahead of and along an insertion axis of the distal tip while positioned within biological tissue of an internal organ of a subject; an optical interface configured to couple the optical probe to an external optical scanning system and to transmit scanning light from the external optical scanning system to the distal tip and return optical signals from the distal tip; and a computing and processing unit configured to: (i) determine, using the structural OCT data, a tissue classification of the region ahead of the distal tip based on depth-resolved optical signal characteristics; (ii) identify one or more vascular structures based on flow-related information derived from the Doppler OCT data; (iii) determine a spatial relationship between the one or more vascular structures and the distal tip, the spatial relationship including at least (a) a distance from the distal tip of the probe to the one or more vascular structures and (b) a size of the one or more vascular structures, wherein the distance is determined by converting optical path length information derived from the depth-resolved OCT data into a physical distance referenced to the distal tip along the insertion axis; (iv) evaluate the spatial relationship in view of the tissue classification under a predefined condition to determine a risk metric associated with advancing the distal tip toward the one or more vascular structures prior to physical contact, and generate guidance information based on the risk metric; wherein the assembly is configured to provide the guidance information in real time to assist positioning of the instrument so as to avoid the one or more vascular structures.
Embodiment 34. The assembly of embodiment 33, wherein the optical probe comprises a GRIN lens disposed at a forward portion of the probe and extending to the distal tip such that a distal end of the GRIN lens defines an imaging interface at the distal tip for forward-view imaging into tissue ahead of the distal tip, and wherein the optical interface comprises an adapter configured to align and stabilize the GRIN lens with respect to the external optical scanning system and to maintain optical alignment for transmission of scanning light and return signals.
Embodiment 35. The assembly of embodiment 33, wherein the optical probe is free of any optical, motorized, or electromechanical scanning component at the distal end such that all optical scanning is performed by the external optical scanning system located proximal to the probe.
Embodiment 36. The assembly of embodiment 33, wherein the optical probe is configured to acquire the depth-resolved optical data from tissue located ahead of the distal tip prior to physical contact between the distal tip and the tissue, such that the acquired data represents subsurface tissue structure at a distance from the distal tip based on optical path length information.
Embodiment 37. The assembly of embodiment 33, wherein the spatial relationship is determined in a coordinate system referenced to the distal tip of the optical probe and aligned with the insertion axis such that distances are measured relative to the distal tip along the insertion direction.
Embodiment 38. The assembly of embodiment 33, wherein the predefined condition is dependent on the tissue classification and defines a risk metric associated with vascular injury as a function of the distance to and the size of the one or more vascular structures.
Embodiment 39. The assembly of embodiment 33, wherein identifying the one or more vascular structures comprises detecting flow-related frequency features from Doppler OCT data, the flow-related frequency features corresponding to motion-induced phase shifts indicative of blood flow.

### Additional Embodiments

While several embodiments have been provided in the present disclosure, it may be understood that the disclosed systems and methods may be embodied in many other specific forms without departing from the spirit or scope of the present disclosure. The present examples are to be considered as illustrative and not restrictive, and the intention is not to be limited to the details provided herein. Various modifications, substitutions, combinations, or alterations may be made without departing from the spirit and scope of the disclosed embodiments.

1. A method for tip-referenced forward-view interventional guidance and comprising:
inserting a probe into biological tissue of an internal organ of a subject, wherein the probe comprises a distal tip;
acquiring depth-resolved structural optical coherence tomography (OCT) data and depth-resolved flow-sensitive Doppler OCT data simultaneously from a region of the biological tissue located ahead of and along an insertion axis of the distal tip while the probe is positioned within the biological tissue and before the probe physically contacts the region;
determining, based on first imaging features of the depth-resolved structural OCT data but not the depth-resolved flow-sensitive Doppler OCT data, a tissue classification of the region relative to surrounding tissues;
identifying a tissue type of the region based on the tissue classification, wherein the tissue type is a tumorous type or another pathological type;
determining, using the depth-resolved flow-sensitive Doppler OCT data but not the depth-resolved structural OCT data, a spatial relationship between the distal tip and one or more vascular structures of the region, wherein the spatial relationship comprises a distance from the distal tip to the one or more vascular structures or a size of the one or more vascular structures, and wherein the distance is based on an optical path length corresponding to a depth position along the insertion axis;
performing, based on the tissue type and a predefined condition, an evaluation of the spatial relationship by comparing the distance and the size to thresholds that vary according to the tissue type to generate a risk metric; and
generating guidance information based on the evaluation, based on the risk metric, and before the distal tip reaches the region, wherein the guidance information suggests adjusting a trajectory or a position of the probe relative to the tissue type and the one or more vascular structures at the distal tip to assist tissue targeting or biopsy guidance.

2. The method of claim 1, wherein determining the tissue classification comprises processing the depth-resolved structural OCT data using a trained deep learning model trained on second imaging features of labeled OCT data corresponding to known tissue types of internal organs, and wherein the second imaging features comprise a signal intensity distribution, an attenuation characteristic, or a morphological feature.

3. The method of claim 1, wherein determining the spatial relationship comprises analyzing Doppler OCT signal variations of the depth-resolved flow-sensitive Doppler OCT data, wherein the Doppler OCT signal variations comprise phase shift features or frequency shift features indicative of moving scatterers corresponding to blood flow.

4. The method of claim 1, further comprising further acquiring the depth-resolved structural OCT data and the flow sensitive Doppler OCT data simultaneously.

5. The method of claim 1, wherein determining the spatial relationship comprises determining the distance relative to a reference position defined at the distal tip by converting optical path length information derived from the depth-resolved structural OCT data and the depth-resolved flow-sensitive Doppler OCT data into a physical distance along the insertion axis such that the distance corresponds to a spatial location ahead of the distal tip.

6. The method of claim 1, wherein the predefined condition is dependent on the tissue type.

7. The method of claim 6, wherein the predefined condition comprises a risk threshold that varies according to the tissue type.

8. The method of claim 1, further comprising generating a risk evaluation result representing a probability or a likelihood of encountering a vascular structure within a predefined distance ahead of the distal tip based on the depth-resolved structural OCT data and the depth-resolved flow-sensitive Doppler OCT data.

9. The method of claim 1, wherein generating the guidance information comprises determining, based on the risk metric, the trajectory in order to reduce a likelihood of intersecting the one or more vascular structures.

10. The method of claim 1, continuously repeating the acquiring, the identifying, the determining, and the generating in real time during the inserting.

11. The method of claim 1, wherein the tumorous type comprises a benign type or a malignant type.

12. The method of claim 11, wherein the guidance information comprises biopsy guidance information based on differentiation between a pathological tissues and a normal tissue.

19. (canceled)

20. A system for tip-referenced forward-view interventional guidance and comprising:
a probe comprising a distal tip and configured to:
be inserted into biological tissue of an internal organ of a subject; and
acquire depth-resolved structural optical coherence tomography (OCT) data and depth-resolved flow-sensitive Doppler OCT data from a region of the biological tissue located ahead of and along an insertion axis of the distal tip while the probe is positioned within the biological tissue; and
a computing and processing unit configured to:
determine, based on first imaging features of the depth-resolved structural OCT data but not the depth-resolved flow-sensitive Doppler OCT data, a tissue classification of the region relative to surrounding tissues;
identify a tissue type of the region based on the tissue classification, wherein the tissue type is a tumorous type or another pathological type;
determine, using the depth-resolved flow-sensitive Doppler OCT data but not the depth-resolve structural OCT data, a spatial relationship between the distal tip and one or more vascular structures of the region, wherein the spatial relationship comprises a distance from the distal tip to the one or more vascular structures or a size of the one or more vascular structures, and wherein the distance is based on an optical path length corresponding to a depth position along the insertion axis;
perform, based on the tissue type and a predefined condition, an evaluation of the spatial relationship by comparing the distance and the size to thresholds that vary according to the tissue type to generate a risk metric; and
generate guidance information based on the evaluation, based on the risk metric, and before the distal tip reaches the region, wherein the guidance information suggests adjusting a trajectory or a position of the probe relative to the tissue type and the one or more vascular structures at the distal tip to assist tissue targeting or biopsy guidance.

21. The system of claim 20, wherein the probe comprise a gradient index (GRIN) lens disposed at and defining the distal tip, wherein the GRIN lens is configured to:
provide forward-view imaging at the distal tip; and
directly interface with the biological tissue, and
wherein the probe is free of any optical scanning component at the distal tip such that the probe is configured to perform optical scanning at a proximal location of the probe.

22. The system of claim 21, further comprising a scanning mechanism located proximate to the probe and configured to perform optical the optical scanning to obtain optical beams, wherein the GRIN lens is further configured to relay the optical beams from the scanning mechanism to the distal tip.

23. The system of claim 22, further comprising an optical fiber, a fiber-optic collimator, or a coupling adapter coupling the GRIN lens and the scanning mechanism.

24. The system of claim 21, wherein the GRIN lens is further configured to further provide the forward-view imaging so that the forward-view imaging aligns with the insertion axis.

25. The system of claim 21, wherein the probe is free of a motorized component or an electromechanical component at the distal tip.

26. The system of claim 21, wherein the GRIN lens has a pitch and a length configured to position an imaging plane at or near the distal tip.

27. The system of claim 20, wherein the probe is further configured to be removably inserted a lumen of a needle, a trocar, a catheter, a cannula, or a biopsy instrument.
